(19) United States
(12) Patent Application Publication
(10) Pub. No.: US 2018/0217144 A1
(43) Pub. Date: Aug. 2, 2018

(54) INHIBITORY IMMUNOGLOBULINS

(71) Applicant: The University of Birmingham, Birmingham (GB)

(72) Inventors: Ian Robert Henderson, Birmingham (GB); Timothy Wells, Birmingham (GB)

(73) Assignee: The University of Birmingham, Birmingham (GB)

(21) Appl. No.: 15/745,959

(22) PCT Filed: Jul. 20, 2016
(86) PCT No.: PCT/GB2016/052200

§ 371 Date: Jan. 18, 2018

Related U.S. Application Data
(60) Provisional application No. 62/194,606, filed on Jul. 20, 2015.

(51) Int. Cl. G01N 33/569; G01N 33/68

(52) U.S. Cl. CPC G01N 33/56911; G01N 33/6854; G01N 2333/21; G01N 2800/52; G01N 2469/20; G01N 2800/12; G01N 2400/50

(57) ABSTRACT

The present invention relates to methods for identifying the presence or elevated levels of IgG2 specific for O-antigen of Gram-negative bacteria in a subject. The method comprises providing a binding agent specific for said IgG2, contacting the binding agent with the sample, allowing the binding agent and IgG2 to form a complex and thereafter directly or indirectly detecting the complex. Also provided are methods for assessing the severity of infection and/or a worsening of a patient's condition. The present invention also relates to isolated O-antigens.

### FIELD OF THE INVENTION

The present invention relates to methods for identifying the presence or elevated levels of IgG2 specific for O-antigen of Gram-negative bacteria in a subject. This may also be indicative of the severity of infection and/or a worsening of a patient's condition, such as airway/lung/bronchiolar tree condition such as non-cystic fibrosis bronchiectasis. It may also be indicative of a Gram-negative infection such as P. aeruginosa infection in a patient.

### INTRODUCTION

Non cystic fibrosis (non CF) bronchiectasis is a pathological condition of lung damage characterized by inflamed, dilated and thick-walled bronchi, and may be localised or diffuse. Conditions predisposing to development of non CF bronchiectasis can include host immune defects, post infective sequelae and defects of mucociliary clearance. The underlying cause however is identifiable only in about 50% of cases¹. It is characterised by chronic production of mucopurulent or purulent sputum, persistent bacterial colonisation and recurrent lower respiratory tract infections. Pseudomonas aeruginosa is isolated in 5-31% of adult patients with non CF bronchiectasis². Colonisation with this organism is associated with poorer quality of life³, and is an independent risk factor for declining lung function in non CF bronchiectasis⁴. It has also been suggested that infection with P. aeruginosa may confer a worse prognosis compared with other pathogens^{5,6}. Once P. aeruginosa colonisation is established, it is difficult to eradicate and often resistant to numerous antibiotics, making management of the condition difficult. Patients with P. aeruginosa colonisation will often require treatment with long term antibiotic therapy.

People suffering from bronchiectasis often succumb to the ‘vicious cycle’ hypothesis where failure of host defence leads to a host-mediated chronic inflammatory response causing further impairment of mucociliary clearance and host defences, thereby amplifying the problem. The interplay between bacterial organisms and host defence represents a frustrated attempt at clearance, leading to excessive inflammation and maintaining the vicious cycle⁷. Therefore, to combat chronic infection effectively an understanding of both the infecting bacteria and the human response to infection is vital.

### SUMMARY OF INVENTION

The present invention is based in part on studies by the present inventors where it has been identified that certain subjects with respiratory infections are identified as being infected with Pseudomonas strains which are resistant to killing by the patient's own serum. It has been observed that the serum is unable to kill the strains due to elevated IgG2 specific for O-antigen.

In the first aspect, there is provided a method for detecting the presence or elevated level of IgG2 specific for O-antigen from Gram-negative bacteria, in a sample from a subject. The method may comprise providing a binding agent specific for said IgG2, contacting the binding agent with the sample, allowing the agent and IgG2 to form a complex and thereafter directly or indirectly detecting the complex. Conveniently, the agent specific for said IgG2 is O-antigen, or a IgG2 specific fragment thereof.

The method may be applicable for detecting the presence and/or initial colonisation of Gram-negative bacteria in a patient.

The method may be applicable for detecting severe or worsening disease, so as to facilitate a clinician in determining an appropriate treatment for the patient.

As infection worsens, the severity of a disease that encourages such infection may also worsen. As such, the presence of O-antigen specific IgG2 may be indicative of, e.g. prognostic of future, reduced lung function. In other words, this may also be indicative of a worsening airway/lung/bronchiolar tree condition such as non-cystic fibrosis bronchiectasis.

Accordingly, in one embodiment the presence of O-antigen specific IgG2 is indicative of the severity of a further condition, such as reduced lung function, a worsening airway/lung/bronchiolar tree condition, or obstructive lung disease. The condition may be bronchiectasis, such as non-cystic fibrosis bronchiectasis. In one embodiment the condition may be cystic fibrosis.

In one embodiment, the method is for determining whether the patient's sample, such as a serum sample has a level of O-antigen specific IgG2 that will inhibit immune-killing of O-antigen containing bacteria. This may also determine the severity of a Gram negative bacterial infection, e.g. a Pseudomonas infection, such as P. aeruginosa, infection in a patient.

The presence of the O-antigen coupled to lipopolysaccharide (LPS) is indicative of the presence of so-called “LPS-smooth” (or simply “smooth”) Gram-negative bacterial strains. Although the presence and type of O-antigen varies between strains and indeed species, the O-antigen is found as the most peripheral unit in the LPS (which, when complete, comprises the O-antigen, the outer and inner core and the transmembrane Lipid A). The LPS, as is well known, is found on the outer membrane of Gram-negative bacteria. So-called “LPS-rough” (or simply “rough”) Gram-negative bacterial strains lack the O-antigen of LPS and present (i.e. display) only the outer and inner core of LPS (attached of course to the transmembrane Lipid A of LPS) on their outer membrane.

Without wishing to be bound by theory, the present inventors have observed IgG2 which is present in the serum of some patients is able to bind O-antigen. In particular, patients with elevated levels of IgG2 which is capable of binding O-antigen, display an impaired immune killing of Gram-negative bacteria. Thus, patients who are infected with smooth Gram-negative bacteria (i.e bacteria which express O-antigen) and who display elevated-levels of IgG2 which is capable of binding O-antigen, display a reduced ability of a patient's immune system to kill the infection-causing Gram-negative bacteria. The inventors have observed that this leads to a more severe or worsening disease state, which may be difficult to control without continued antibiotic administration and/or prevent or reduce infection based upon the generation of an immune response to O-antigen administration. Such knowledge may allow the clinician to adopt specific therapeutic strategies designed to address such conditions.

The present invention is concerned with identifying patients with elevated O-antigen specific IgG2. The present invention is applicable to any O-antigen from any Gram negative bacterial species which are typically associated with infection in humans or animals. This includes the proteobacteria (such as Escherichia coli (E. coli), Salmonella, Shigella, and other Enterobacteriaceae, Pseudomonas (especially Pseudomonas aeruginosa), Moraxella, Helicobacter, Stenotrophomonas, Bdellovibrio, and Legionella. Other examples of relevant Gram-negative bacteria include Neisseria; Hemophilus; Ralstonia, Klebsiella, Acinetobacter, Proteus, and Serratia.

There is also provided, therefore, a method for assessment of the severity or worsening of disease which is associated with or caused by a Gram-negative infection, such as P. aeruginosa infection, comprising determining the presence or elevated levels of IgG2 capable of binding said O-antigen in a sample or samples, the presence or elevated levels of O-antigen specific IgG2 being indicative of an increased severity or worsening of disease associated with or caused by a Gram negative bacteria, such as P. aeruginosa strains displaying said O-antigen. The increased severity or worsening of a condition may be a reduced tissue or lung function, a worsening airway/lung/bronchiolar tree condition, or obstructive lung disease. The condition may be Bronchiectasis, in particular non-cystic fibrosis Bronchiectasis. The condition may be cystic fibrosis.

It is appreciated that there may be more than one strain causing the infection of the patient. Where more than one strain is present, all such strains or only a selection of the strains may express O-antigen.

In one embodiment the infection is associated with P. aeruginosa. It will be appreciated that the majority of P. aeruginosa infections are found in the airway (especially the lungs and trachea). Whilst the infection may be a P. aeruginosa infection of the airway, it may for example be a P. aeruginosa infection of the bronchial tree.

The patient may have obstructive lung disease, especially bronchiectasis. Such conditions render the patient increasingly susceptible to infection by P. aeruginosa.

The method may be indicative of chronic colonisation with P. aeruginosa.

The patient may be human.

Also provided is a method of determining the efficacy of treatment for a smooth Gram negative infection, such as a smooth P. aeruginosa infection in an subject comprising determining in samples from the subject, whether the levels of smooth Gram-negative bacteria which express O-antigen capable of binding IgG2 specific for said O-antigen has decreased after the treatment.

Conveniently detection of IgG2 and/or O-antigen specific IgG2 may be carried out on any suitable biological sample. The biological sample may be any appropriate fluid sample obtained from the subject. For example, the fluid sample may comprise at least one of: urine; saliva; blood and blood fractions such as plasma, or serum; sputum; semen; mucus; tears; a vaginal swab; a rectal swab; a cervical smear; a tissue biopsy; a urethral swab and a lavage. The biological sample may depend on the site of infection. For example, if the infection is a lung infection, a suitable sample may be serum, sputum, lavage or biopsy sample. The most appropriate sample type can be determined by the skilled clinician faced with a particular subject and the type of infection.

Presence of Gram-negative bacteria which display O-antigen, may be carried out by a variety of techniques known to the skilled reader. For example O-antigen specific antibodies, known in the art may be employed in assays to detect bacteria displaying O-antigen. Many suitable antibody/O-antigen detection methods are known to the skilled addressee. Examples include radioimmunoassay (RIA), enzyme-linked immunosorbent assay (ELISA), Western blotting, flow cytometry, electrochemiluminescent assays, plasmon and surface enhanced resonance assays.

Alternatively, the presence of O-antigen may be detected by way of a histological technique, where the bacteria are initially isolated and/or grown so that they may be stained and/or visually studied in order to determine whether or not the bacteria are displaying O-antigen.

It is possible to detect O-antigen by mass spectrometry type techniques, where the LPS component is released from the cell surface and based on the mass of the LPS molecule it is possible to determine whether or not O-antigen is present. An example of this is described in WO2014035270, to which the skilled reader is directed and the entire contents of which are incorporated herein by way of reference.

It is also possible to simply detect the nucleic acid within a particular bacteria, which encodes the enzymes required for O-antigen synthesis and/or coupling to the LPS core. Identifying such genes within a bacterial strain is indicative that O-antigen is present on the bacterial strain. EP0904376, for example, identifies the genes/proteins from Pseudomonas aeruginosa which are associated with the synthesis and assembly of O-antigen (the skilled reader is directed to this document, the entire contents of which are incorporated herein by way of reference). Simple nucleic acid based tests may employ the use of nucleic acid molecules which may be used as primers to amplify a nucleic acid molecule associated with the aforementioned enzymes, for example using polymerase chain reaction (PCR) or other amplification based or specific hybridization assays well known to the skilled reader and described for example in, SAMBROOK and RUSSELL: “Molecular Cloning: A Laboratory Manual”, vol. 3, 2001, COLD SPRING HARBOR LABORATORY PRESS, to which the skilled reader is directed, the entire contents of which are incorporated herein by way of reference.

In a preferred embodiment, the presence of O-antigen is detected by an immunological method, such as a competitive or non-competitive immunoassay, preferably using a solid-phase antibody, an ELISA or ELISPOT assay.

Methods of detecting or antibodies that recognise LPS from P. aeruginosa are provided in WO2002020619 which relates to human antibodies produced in non-human animals that specifically bind to P. aeruginosa Lipopolysaccharide (LPS) that might be useful herein. It further provides methods for making the antibodies in a non-human animal, expression of the antibodies in cell lines including hybridomas and recombinant host cell systems. Also provided are kits and pharmaceutical compositions comprising the antibodies and methods of treating or preventing pseudomonas infection by administering to patient the pharmaceutical compositions.

WO2005056601 provides human antibodies produced in non-human animals that specifically bind to lipopolysaccharide (LPS) from strains Fisher Devlin (International Serogroups) It-2 (011), It-3 (02), It-4 (01), It-5 (010), It-6 (07), PA01 (05), 170003 (02), IATS016 (02/05), and 170006 (02). It further relates to methods for making the antibodies in a non-human animal, expression of the antibodies in cell lines including hybridomas and recombinant host cell systems. Also provided are kits and pharmaceutical compositions comprising the antibodies and methods of treating or preventing pseudomonas infection by administering to a patient the pharmaceutical compositions herein.

Other methods of detecting immunoglobulins in particular are known. Other methods for detecting the O-antigen of LPS from P. aeruginosa are known such as in Ansorg et al (hereby incorporated by reference) where slide coagulation tests can identify ‘smooth’ strains of P. aeruginosa.

Typically, IgG2 may be detected using an antibody which is specific for IgG2. Such an antibody may be obtained commercially from Life Technologies, for example mouse anti-human IgG2 (05-3522). Any IgG2 may be initially be captured by using purified O-antigen which is known to be capable of binding the O-antigen specific IgG2. O-antigen specific IgG2 levels can be determined by detecting the titre of IgG2 that can bind one- or multiple purified O-antigens. Typically, purified O-antigen will be immobilised and then contacted with the patient sample. By “immobilised”, the purified O-antigen may be bound to a suitable substrate such as sepharose, polylysine, polymyxin B, magnetic beads and plastics. A labelled IgG2 specific antibody—e.g. conjugated to an enzyme that allows detection eg. Alkaline phosphatase or horseradhish peroxidase is then applied to the assay. In the presence of the correct substrate the titre of anti-O-antigen IgG2 can be determined by the level of reaction to the substrate.

The term “elevated” in terms of IgG2 levels is understood to relate to IgG2 when present in blood or more preferably plasma or serum. Thus, typically a level of IgG2 will be determined from a sample of blood, serum or plasma. Elevated in the context of the present invention is understood to mean higher than a mean or normal range value of IgG2 levels, which are capable of binding to the O-antigen from a Gram negative bacteria identified from a population of subjects wherein the serum/plasma from such subjects is capable of killing said smooth Gram negative bacteria. “Higher” in this context is taken to mean at least 10%, such as 15%, 20%, 25%, or 40% or more, higher than an upper normal range value, or at least 50%, 75%, 100%, 200%, 250%, or 300%, or more than a mean IgG2 titre value as determined from a population of subjects which have serum/plasma which is capable of killing the infective gram negative bacterial strain or strains.

Such mean or range values may be determined empirically, or may be predetermined and disclosed for use by a clinician, For example, predetermined reference values may be disclosed or otherwise published in relation to specific smooth Gram negative bacterial infection and/or associated medical condition. Thus, a clinician faced with a new subject presenting with the specific infection and/or medical condition, may simply determine a Gram negative specific IgG2 plasma/serum level and compare this against the disclosed predetermined reference value, in order to ascertain whether or not the subject has an elevated smooth Gram negative specific IgG2 plasma/serum level.

By way of example, this is described in the detailed description, where 11 P. aeruginosa infected bronchiectasis subjects where analysed in order to determine P. aeruginosa specific IgG2 serum levels for all 11 subjects. Of the 11 subjects, 8 subjects had serum which was capable of killing the infective P. aeruginosa and 3 subjects had serum which was incapable of killing the infective P. aeruginosa. Of the 8 subjects who had serum which was capable of killing the infective P. aeruginosa, their IgG2 levels specific to the O-antigen of the infective P. aeruginosa were identified and a normal or control IgG2 titre range and mean level obtained. The normal/control IgG2 titre range was approximately 200-2600 with a mean of approximately 1100. In the context of that particular example, an elevated level may therefore be seen as being above the upper range value of 2600 or above the average of 1100. Thus an elevated titre level in the context of a bronchiectasis patient with a P. aureuginosa Infection may be above about 2800. The elevated IgG2 titre levels, including standard error values, from the 3 subjects who had serum which was incapable of killing the infective Pseudomonas aeruginosa were all above 4000.

Thus, in a further aspect there is provided a method for detecting inhibitory immunoglobulin molecules, typically IgG2 molecules from a patient, the method comprising:

mixing a sample of the patient's serum or plasma with purified O-antigen which is capable of binding inhibitory immunoglobulin molecules, in order to allow any O-antigen specific immunoglobulin moieties which are present in the patient's serum or plasma sample, to bind to the purified O-antigen; and detecting any immunoglobulin moieties which are bound to the purified O-antigen. Typically the inhibitory immunoglobulin molecules comprise or consist essentially of IgG2 molecules.

The method may further comprise confirming whether or not the patient's serum is capable of killing of the Gram negative bacteria infecting the patient and hence whether or not the inhibitory immunoglobulin molecules are present at a sufficiently high-enough concentration to prevent serum killing. It is also possible to identify a threshold level above which it may be expected that a level of inhibitory immunoglobulin molecules will be sufficient to prevent serum killing of infecting gram negative bacteria. In this manner, it would not be necessary to test a patient's serum/plasma O-antigen specific immunoglobulin levels. The skilled addressee can easily determine a suitable threshold level by looking at a population of patients with a particular condition and/or smooth Gram negative infection. For each patient their O-antigen specific immunoglobulin levels and whether or not their serum is capable of inhibiting serum killing will be determined. This allows the skilled addressee to determine an average or threshold level for O-antigen specific immunoglobulins, above which it may be expected that a patient's serum would not be capable of killing all of the infective smooth Gram negative bacteria and hence that the infection may be considered, for example a severe one and/or one which would lead to a worsening of the patient's condition. As mentioned above in relation to the first aspect, the threshold value may be determined empirically, or may be predetermined and disclosed for use by a clinician when faced with a particular infection and/or condition.

For example, the skilled addressee may take a group (e.g. at least 10, 15, 20, 25, 50 or more—the more the better) of patients with a particular smooth Gram negative infection. Their O-antigen specific immunoglobulin levels can be determined as well as the ability of each patient's serum to be able to kill the infective Gram negative bacteria in an in vitro test, as described herein. This will allow the skilled addressee to ascertain from the collective data, when O-antigen specific immunoglobulin levels are and are not sufficient to prevent serum killing of the infective gram negative bacteria. The skilled reader may then, as described previously, identify a range and/or average level for O-antigen specific immunoglobulin levels which are expected to be sufficient to permit serum killing of the infective smooth Gram negative bacteria. O-antigen specific immunoglobulin levels above such a range and/or average threshold value would be expected to prevent serum killing. The threshold value may be set to be, for example at least 10%, such as 15%, 20%, 25%, or 40% or more, higher than an upper range value for immunoglobulin levels which are insufficient to prevent serum killing, or, for example, at least 50%, 75%, 100%, 200%, 250%, or 300%, or more than a mean immunoglobulin level from serum samples which are insufficient to prevent serum killing.

In a further aspect there is provided a method of obtaining said isolated O-antigen(s), the method comprising: providing a bacterial strain or strains which express O-antigen capable of specifically binding an inhibitory immunoglobulin(s), growing the bacterial strain(s) and obtaining the O-antigen.

The inventors have invented an O-antigen isolation method, which, surprisingly, can be used to isolate pure O-antigen (i.e. the O-antigen may be substantially free of other cell surface and/or LPS components).

Typically the O-antigen may be purified from the bacterial strain(s). Such purification may include the use of acetic acid. The bacterial strain(s) may be pelleted, using procedures which are known to those skilled in the art (for example centrifugation). The acetic acid may comprise a concentration of at least 0.05, 0.1, 0.2, 0.5, 1 or 2%. The bacterial pellet may be resuspended in media comprising at least 1% acetic acid. The media may comprise at least 2% acetic acid. The method may further comprise incubating the resuspended bacterial strain(s) for at least 1, 2, 3 or 4 hours at a temperature of at least 50, 60, 70, 80, 90 or 100° C. The method may further comprise a centrifugation step. The O-antigen may be substantially free of other cell surface and/or LPS components.

The method may further comprise removing contaminants by protein (e.g. proteinase treatment) and/or DNA and/or RNA degradation (e.g. nuclease treatment). The skilled addressee will be aware of standard contaminant removal processes. The method may further comprise purification using phenol extraction followed by a final exchange and condensation into water, with optional filtering.

The purified O-antigen may be from a single serotype or multiple serotypes, providing that the one or more O-antigen serotypes are from smooth Gram negative strains which have previously been identified from infected patients as giving rise to serum which contains levels of inhibitory immunoglobulin molecules, such as IgG2, which are capable of preventing serum killing of the smooth Gram negative bacteria. Serotyping of O-antigens may be carried out by PCR, genome sequencing or using specific serotype antibodies.

In a further aspect there is provided isolated O-antigens for use in a method as defined herein. Said isolated O-antigens may be present in a mixture comprising two or more isolated O-antigens, such as 2, 3, 4, 5, 6, 7, 8, 9 or more separate O-antigens of different serotype. The mixture may comprise 3 or more separate O-antigens of different serotype. The mixture may comprise all known identifiable O-antigens of different serotype. Said isolated O-antigens are understood to be O-antigens to which inhibitory immunoglobulin molecules of the present invention are capable of specifically binding. Isolated is understood to relate to purifying the O-antigen from the cell surface and other LPS components, as described herein. The phrase “isolated O-antigen” may thus define purified O-antigen which is substantially free of other cell surface and/or LPS components. There is also provided such isolated O-antigens bound to a suitable substrate such as sepharose, polylysine, polymyxin B, magnetic beads and plastics. By “bound” it will be understood that the O-antigens are attached to the suitable substrate, for example by covalent or electrostatic interaction. Thus, the bound O-antigen is not a product of nature. The O-antigen may be formalin or paraformaldehyde-treated.

A mixture comprising two or more isolated O-antigens may be used in a multiplex diagnostic assay. Advantageously, the use of the isolated pure O-antigens removes false positive results. In addition, the use of a mixture of at least two isolated O-antigens of different serotype means that false negatives are not missed; the mixture may provide a universal test which may be used on any patient and/or sample. This provides excellent specificity. Moreover, the use of a multiplex assay reduces time and cost compared to multiple individual assays.

An inhibitory immunoglobulin is an immunoglobulin molecule which is capable of specifically binding an O-antigen serotype which has been identified as being associated with a patient's serum which is not capable of successfully killing the infective smooth Gram negative bacteria. Such an inhibitory immunoglobulin may or may not be capable of binding other O-antigen serotypes. Although other classes of immunoglobulin are envisaged, such as IgA, IgE, IgM and so forth, the inhibitory immunoglobulin being detected typically includes IgG such as the IgG subclass 2 (IgG2). IgG molecules have two heavy chains and two light chains. The skilled person will understand that the the two heavy chains are linked to each other by disulphide bonds and each heavy chain is linked to a light chain by a disulphide bond. It will be appreciated that the binding of an inhibitory immunoglobulin may be indicative of the presence of smooth Gram negative bacteria associated with severe disease or worsening disease.

As the skilled person will be aware, each immunoglobulin class has specialised functions and a unique distribution. Typically, the IgG2 subclass 2 (IgG2) is associated with the functions of neutralisation, activation of the complement system and opsonization. IgG2 may be associated with an extravascular distribution.

The inventors have, in some instances, observed elevated IgA levels in addition to elevated IgG2 levels. Thus, the inhibitory immunoglobulin molecules or moieties may comprise IgG2 and IgA. The aspects of the present invention may thus comprise detecting the presence or elevated level of IgG2 and IgA specific for O-antigen from Gram-negative bacteria.

Without wishing to be bound by theory, the inventors believe that the IgA may act synergistically with the IgG2 to promote inhibition of immune-killing of O-antigen containing bacteria.

Elevated in the context of IgA is understood to mean higher than a mean or normal range value of IgA levels, which are capable of binding to the O-antigen from a Gram negative bacteria identified from a population of subjects wherein the serum/plasma from such subjects is capable of killing said smooth Gram negative bacteria. “Higher” in this context is taken to mean at least 10%, such as 15%, 20%, 25%, or 40% or more, higher than an upper normal range value, or at least 50%, 75%, 100%, 200%, 250%, or 300%, or more than a mean IgA titre value as determined from a population of subjects which have serum/plasma which is capable of killing the infective gram negative bacterial strain or strains.

Such mean or range values may be determined empirically, or may be predetermined and disclosed for use by a clinician, For example, predetermined reference values may be disclosed or otherwise published in relation to specific smooth Gram negative bacterial infection and/or associated medical condition. Thus, a clinician faced with a new subject presenting with the specific infection and/or medical condition, may simply determine a Gram negative specific IgA plasma/serum level and compare this against the disclosed predetermined reference value, in order to ascertain whether or not the subject has an elevated smooth Gram negative specific IgA plasma/serum level.

In a further aspect there is provided a substrate comprising one or more of the aforementioned isolated O-antigens bound thereto. There is also provided a kit comprising said isolated O-antigen(s) and/or substrate comprising said isolated O-antigen(s) bound thereto.

Conveniently, the presence of any bound inhibitory immunoglobulin moieties may detected by an immunological method using an antibody or antibodies (optionally labeled, for example by alkaline phosphatase or peroxidase) which is/are capable of specifically binding to the bound inhibitory immunoglobulin molecules. Typical assays include a competitive or non-competitive immunoassay, preferably using a solid-phase antibody, an ELISA or ELISPOT, well known to the skilled addressee. In an embodiment there is provided a sandwich assay which comprises O-antigen bound to a substrate for capturing any of said inhibitory immunoglobulins and an antibody or antibodies (optionally labeled, for example by alkaline phosphatase or peroxidase) which is/are capable of specifically binding to the O-antigen bound inhibitory immunoglobulin molecules. The labeled antibody can be detected by using an appropriate substrate for the labeled enzyme, which generates a signal, such as a coloured or fluorescent product, following enzyme reaction. A useful description of ELISA assays and reagents may be found in the Thermo Scientific Pierce Assay Development Technical Handbook, available from Thermo Scientific, to which the skilled reader is directed.

It will be appreciated that the terms “specific” and “recognises” are used interchangeable herein and refer to the ability of an antibody to bind with high affinity to a particular target molecule, via strong interactions between the CDR(s) of the antibody and the epitope on the target.

### DETAILED DESCRIPTION OF THE INVENTION

The present invention will now be further described with reference to the following examples and figures which show:

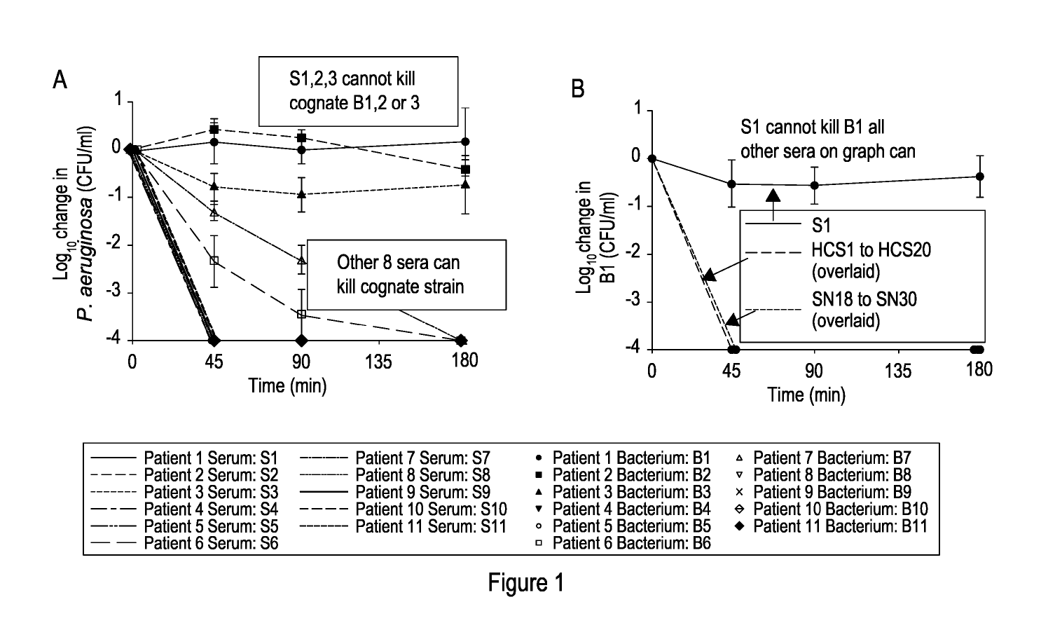

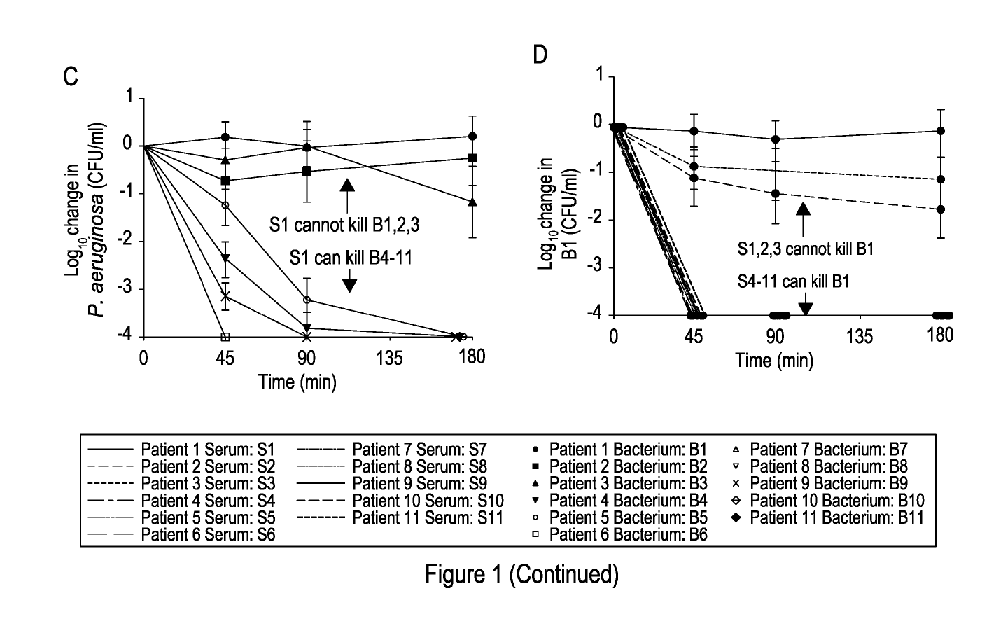

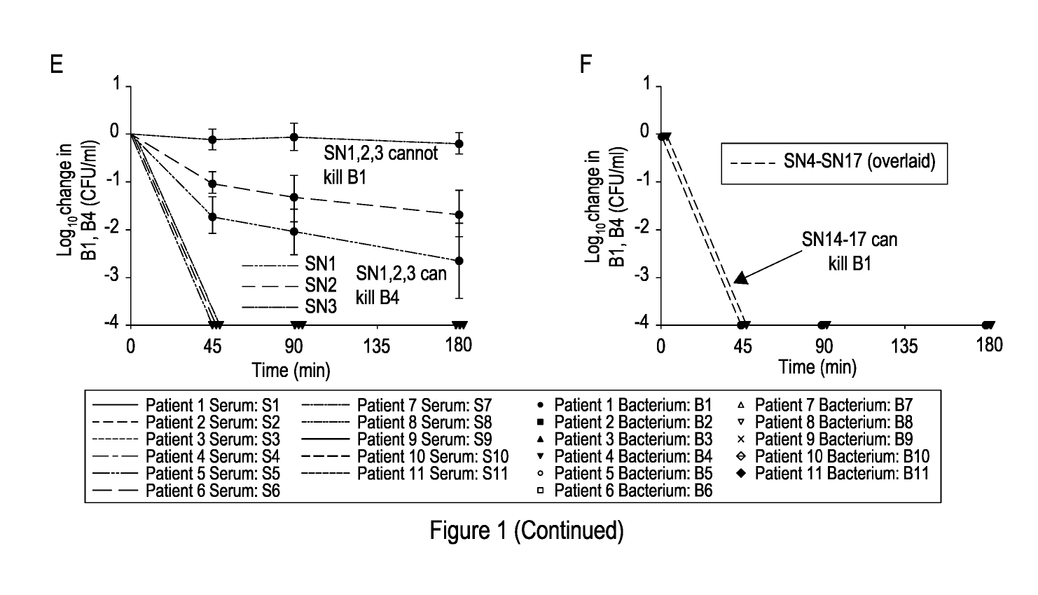

FIG. 1 shows: Identification of patients with impaired serum killing. (A) Killing curves of P. aeruginosa strains isolated from bronchiectasis patients with their autologous serum at 45, 90, and 180 min. Negative values correspond with a decrease in viable P. aeruginosa compared with initial concentration. (B) Killing of B1 by sera taken from 20 healthy people at 45, 90, and 180 min. Killing of B1 by sera from patients with bronchiectasis but without P. aeruginosa colonization (SN18-SN30) is also shown. The curves depicting killing by HCS1-HCS20 and SN18-SN30 are overlaid to simplify. (C) Killing curves of all strains (B1-B11) by serum (S1). (D) Killing curves of P. aeruginosa strain B1 by patient serum (S1-S11). (E) Killing curves of B1 and B4 by sera SN1, 2, and 3 (SN1-3). (F) Killing curves of B1 and B4 by sera SN4-SN17. The curves depicting killing by SN4-SN17 are overlaid to simplify graphs. For all serum bactericidal assays, error bars represent the mean±SD for a minimum of three independent experiments.

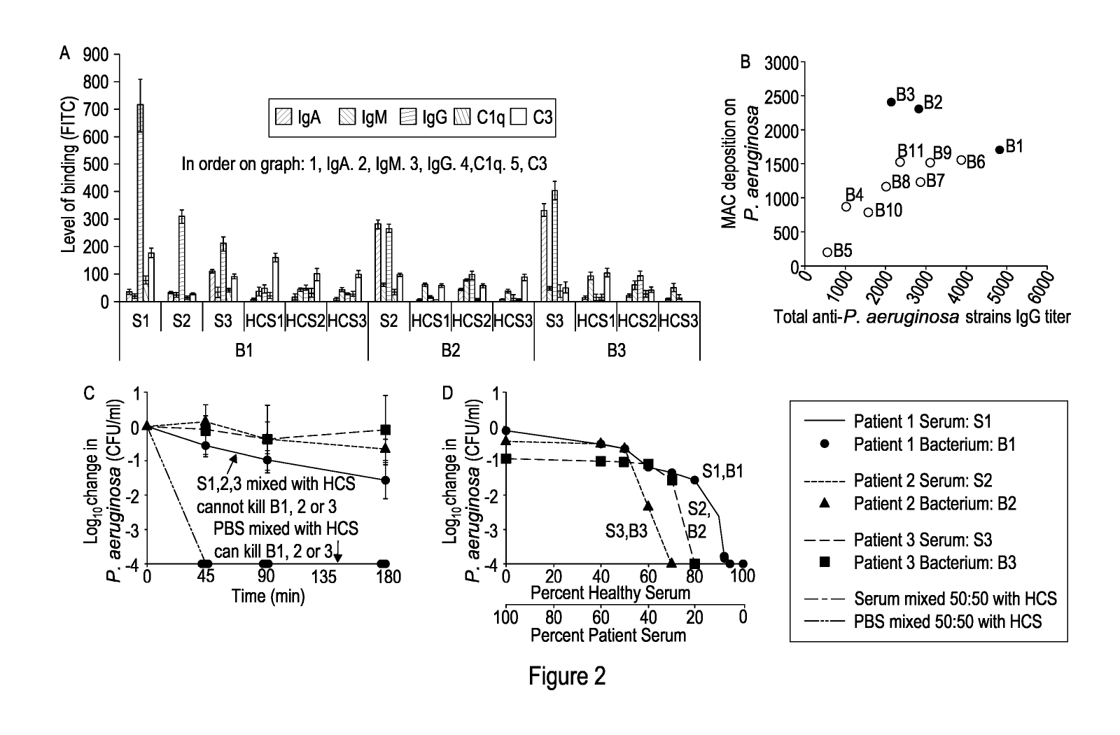

FIG. 2 shows: Impaired serum is caused by a blocking factor, not a lack of antibody or complement. (A) Binding of specific IgG, IgM, IgA antibodies and C1q and C3 complement factors from indicated patient serum to B1, B2, and B3. Each strain was tested with autologous serum and at least three separate healthy controls. Data are representative of three independent experiments. (B) Serum titers of P. aeruginosa-specific IgG compared with C5b-9 MAC deposition on autologous strains. (C) Inhibition of HCS-mediated killing of strains B1-3 with a 50:50 mix of HCS and autologous patient sera (S1-3). Dashed lines represent HCS mixed 50:50 with buffer. (D) Killing of P. aeruginosa strains B1-3 at 180 min by mixed sera consisting of different percentages of HCS mixed with autologous serum. For all data, error bars represent the mean±SD for a minimum of three independent experiments.

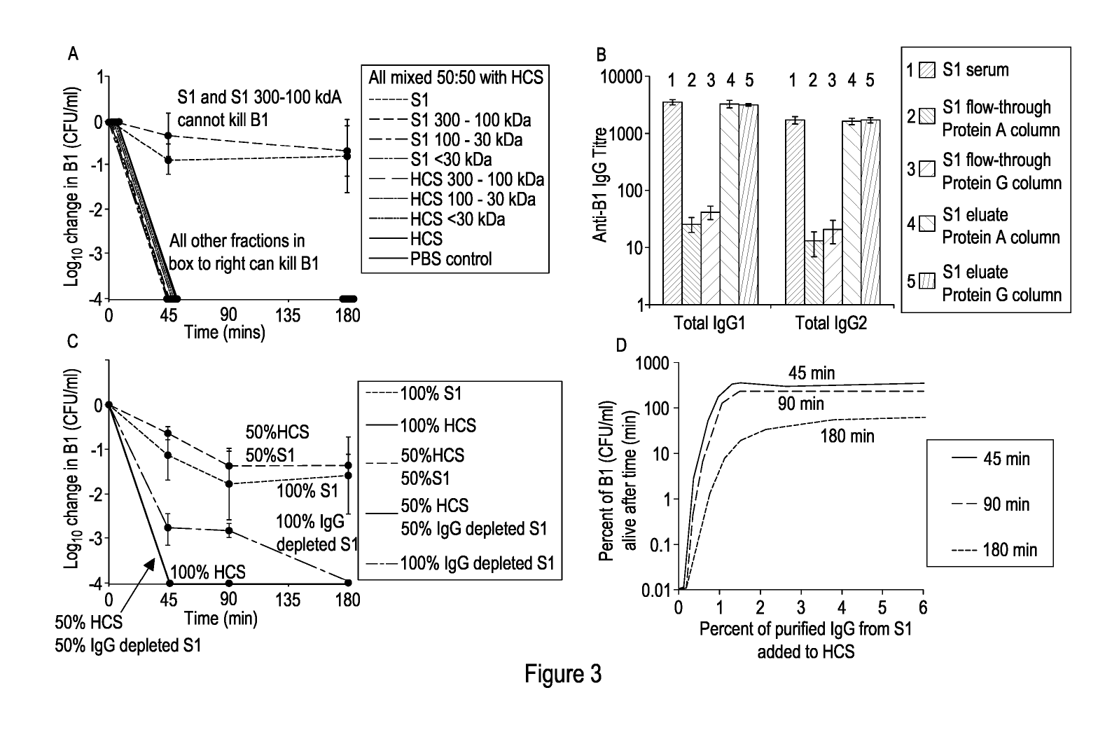

FIG. 3 shows: IgG inhibits serum-mediated killing. (A) Killing of B1 by HCS mixed 50:50 with S1 and HCS fractionated into indicated size ranges. The killing curves for several fractions overlap and have been separated to allow for visualization. Data are representative of three independent experiments. (B) Titer of total IgG1 and IgG2 of S1 serum before and after passing through Protein A and G columns. Data are representative of two independent experiments. (C) Killing curves of P. aeruginosa strain B1 by serum S1. Killing was measured for S1 depleted of antibody using a Protein G column, before and after addition of HCS (50:50). Data are representative of three independent experiments. (D) IgG purified from S1 using a Protein G column was resuspended in PBS to the same volume of serum loaded on the column. Purified IgG was added to HCS in indicated concentrations and measured for its ability to kill B1 at 45, 90, and 180 min. Data are representative of three independent experiments. Error bars represent the mean±SD.

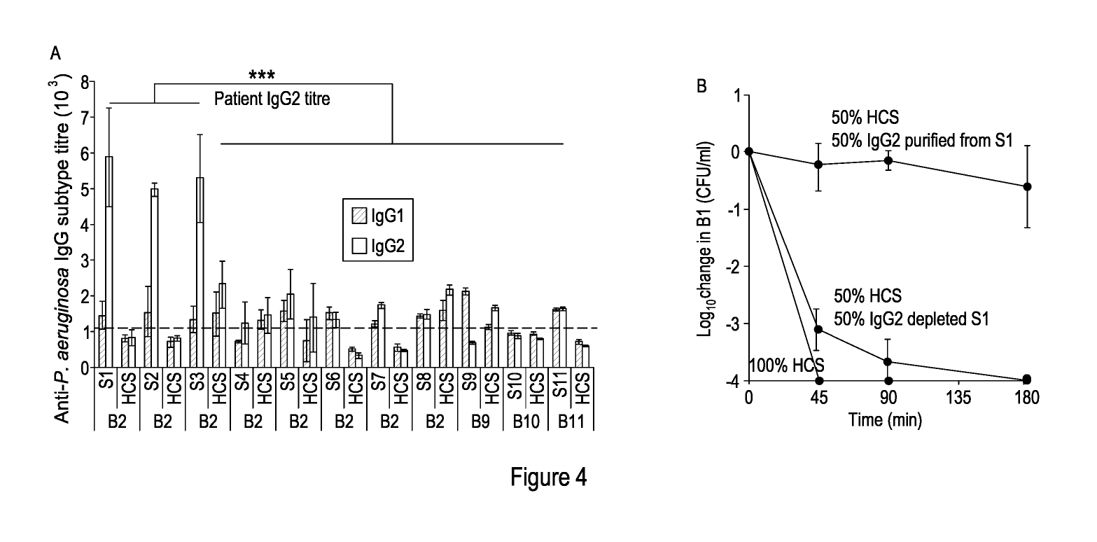

FIG. 4 shows: IgG2 inhibits serum-mediated killing. (A) Titers of IgG1 and IgG2 isotypes in patient serum and HCS that is specific to infecting strains. Dashed line indicates the median IgG2 titer from sera S4-S11. Data are representative of three independent experiments. ***, P<0.001. (B) Killing of B1 was measured with S1 depleted of IgG2 mixed with HCS or with a mixture HCS supplemented with IgG2 purified from S1 or HCS alone. For all data, error bars represent the mean±SD for a minimum of three independent experiments.

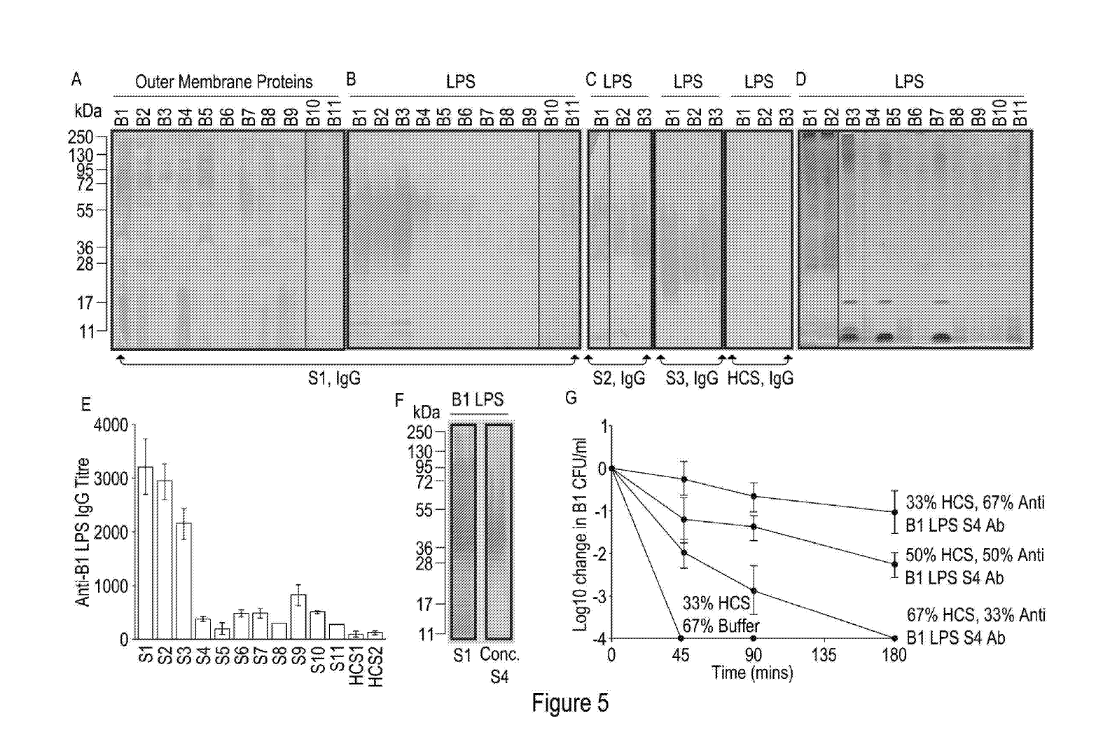

FIG. 5 shows: Inhibitory antibody recognizes P. aeruginosa LPS and is dose dependent. (A) Western blot of outer membrane protein fractions obtained from B1-B11 probed with S1 as a primary antibody and anti-human IgG as the secondary antibody. (B) Polysaccharide-only preparations from P. aeruginosa isolates analyzed by Western blot probed with S1 serum as a primary antibody and anti-IgG as a secondary antibody. (C) Western blot of LPS purified from B1, B2, and B3 probed with S2, S3, or HCS as the primary antibody and anti-human IgG as the secondary antibody. S2 and S3 have high LPS-specific IgG response. In contrast, no anti-LPS IgG is detected in HCS. All Western blots (A-C) are representative of three independent experiments. (D) Polysaccharide-only preparations from P. aeruginosa isolates are analyzed by silver stain. Data are representative of six independent experiments. (E) Patient serum IgG titer specific for LPS isolated from B1 determined by ELISA. LPS was isolated from P. aeruginosa strain B1 and attached to a 96-well plate. ELISA was performed with dilutions of patient or healthy sera and anti-human IgG conjugated to alkaline phosphatase. Error bars represent mean±SD for three independent experiments. (F) Antibodies specific for B1 LPS were purified and concentrated from S4. S1 and anti-LPS antibodies concentrated from S4 were used as primary antibodies in a Western blot against B1 LPS. Data represents three independent experiments. (G) Killing curve of B1 with anti-LPS antibodies concentrated from S4 mixed with HCS. HCS serum similarly diluted with buffer is used as a control. Error bars represent mean±SD for three independent experiments.

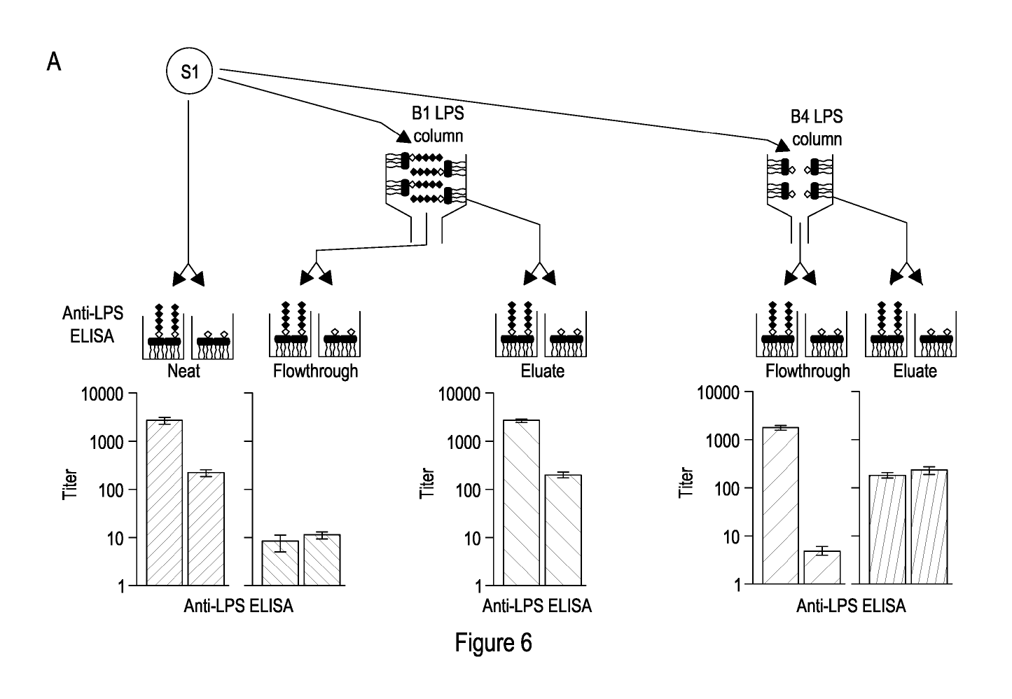

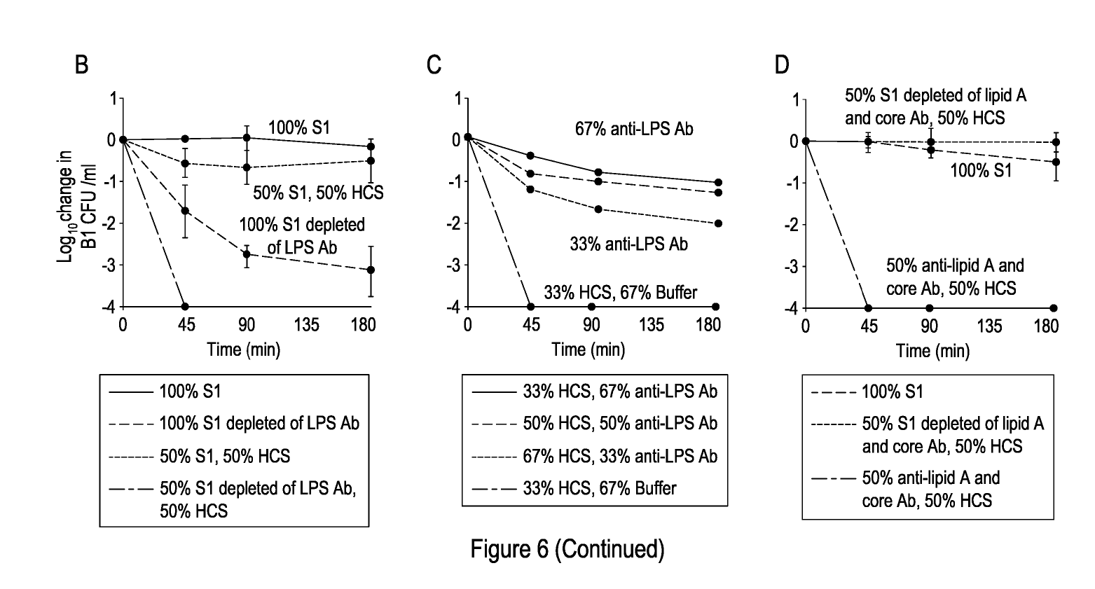

FIG. 6 shows: Inhibitory antibodies are specific for the O-antigen. (A) Schematic of treatment of S1 serum and resultant IgG titers specific to LPS purified from either B1 (with O-antigen) or B4 (without O-antigen). S1 titers against the two LPS extracts are measured after no treatment, passage through, or elution from a column containing purified B1 LPS or passage through or elution from a column containing B4 LPS. In each case, all column fractions were resuspended in PBS to the same volume of serum added to the column. (B) Killing curve of P. aeruginosa B1 treated with S1 or S1:HCS (50:50) depleted of anti-LPS antibodies. (C) Killing curve of B1 after incubation with anti-LPS antibodies purified from S1 and mixed with HCS at different concentrations. (D) Killing curve of P. aeruginosa B1 treated with HCS mixed with either S1 depleted of antibodies to the lipid A and core oligosaccharides of LPS or antibodies that recognize lipid A and core oligosaccharide. For all data, error bars represent the mean±SD of three independent experiments.

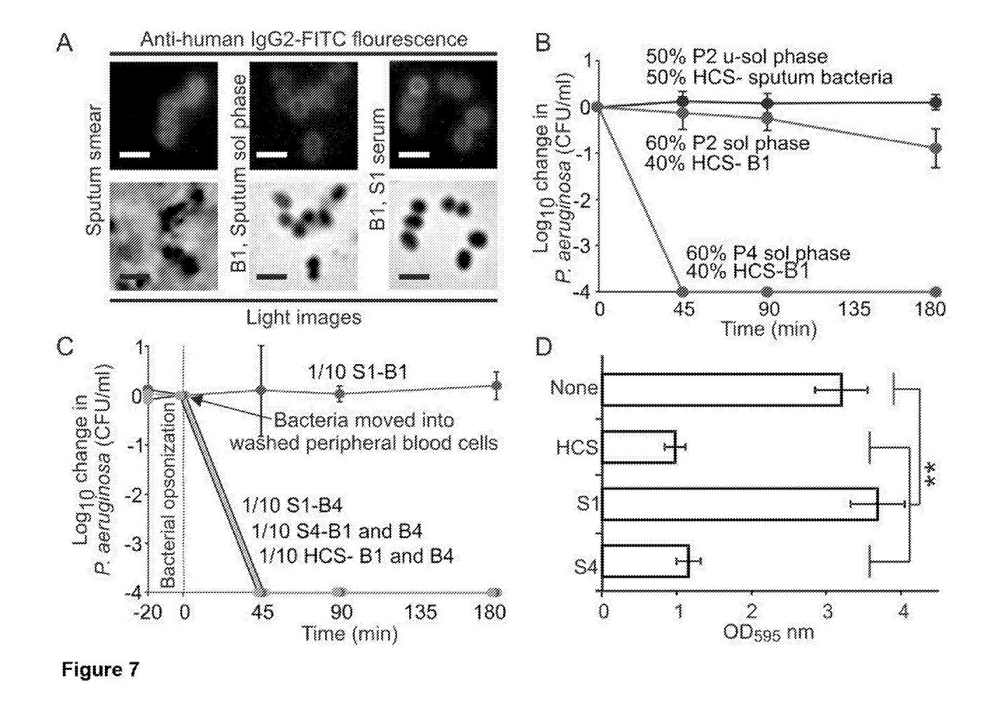

FIG. 7 shows: Significance of inhibitory antibodies in vivo. (A) Immunofluorescence labeling of bacteria present in sputum with anti-human IgG2-FITC (top left). Immunofluorescence labeling of cultured B1 bacteria with sol-phase sputum (top middle) or patient serum (top right) used as the source of primary antibody and anti-human IgG2-FITC. Bottom images are corresponding light images. Bar, 2 μm. (B) Killing curves for bacteria already present in P2 sputum after mixing the unfiltered sol phase with HCS 50:50. Killing curves for B1 with a 40:60 mix of HCS and sterile sol phase sputum isolated from either P2 or P4 are also shown. Error bars represent the mean±SD of three independent experiments. (C) Killing curves for B1 or B4 by washed peripheral blood cells after 20-min opsonization with a 1/10 dilution of HCS, S1, or S4. Negative values correspond with a decrease in viable P. aeruginosa compared with initial concentration. Error bars represent the mean±SD of three independent experiments. (D) Biofilm formation of B1 after overnight growth followed by no treatment or exposure to HCS, S1, or S4 serum for 2 h. Biofilm formation was examined in polystyrene microtiter plates. Error bars represent the mean±SD of 16 independent experiments. **, P<0.01.

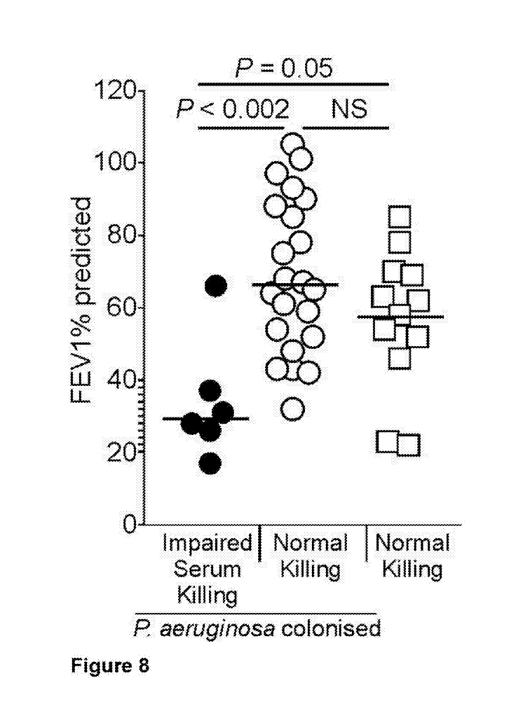

FIG. 8 shows: Inhibitory IgG2 antibody is associated with poor lung function. (A) Comparison of FEV1% predicted values for patients from two non-CF bronchiectasis cohorts that are colonized with P. aeruginosa and display inhibition of serum mediated killing (⊐), patients who are colonized with P. aeruginosa and display normal serum-mediated killing (⊐), or patients who are not colonized with P. aeruginosa (⊐). The horizontal bars represent the median for each group. FEV1% scores represent mean of three independent measurements. *, P<0.05; **, P<0.01.

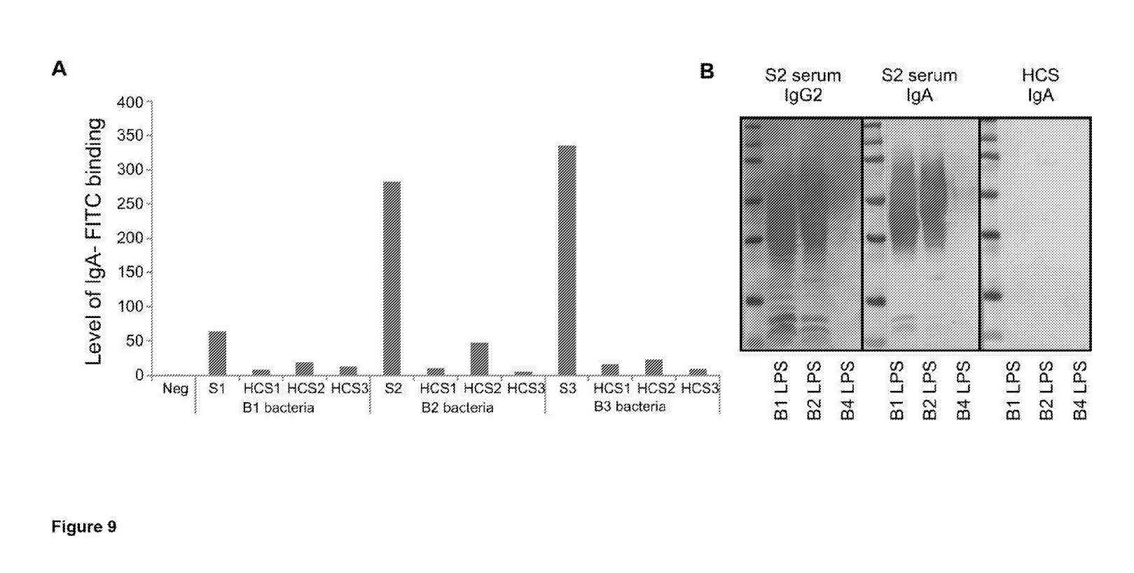

FIG. 9 shows: IgA directed towards O-antigen is also elevated in some patients. (A) Binding of specific IgA antibodies from indicated patient serum to B1, B2, and B3. Each strain was tested with autologous serum and at least three separate healthy controls. Data are representative of three independent experiments. (B) Western blot of LPS purified from B1, B2, and B4 probed with S2 or HCS as the primary antibody and anti-human IgG2 or IgA as the secondary antibody. S2 has high LPS-specific IgG2 and IgGA response. In contrast, no anti-LPS IgA is detected in HCS. All Western blots are representative of three independent experiments.

### MATERIALS AND METHODS

Patient details, strains, and samples. Bronchiectasis patients with and without chronic P. aeruginosa colonization were identified and confirmed by CT scan. Eleven bronchiectasis patients with chronic P. aeruginosa colonization were identified. P. aeruginosa was isolated by sputum culture on chocolate blood agar and Pseudomonas isolation agar and subsequently cultured in Luria broth. Serum was collected from each patient and 20 healthy individuals. Each patient (P), their isolated bacterium (B), and serum (S) were assigned the same number; patient P1, with serum S1, is colonized by P. aeruginosa B1 (Table 1). In the absence of a widely recognized disease severity index in bronchiectasis, the degree of lung function impairment was evaluated using forced expiratory volume in 1 s (FEV1) as a percent predicted of a normal FEV1. This work was performed in compliance with the human ethical approval guidelines granted by the Birmingham Ethics Committee (code RRK3404) and Newcastle and North Tyneside Research Ethics committee (code 12/NE/0248). Additional serum samples were obtained from patients with bronchiectasis regardless of whether they had P. aeruginosa colonization or not. These samples were from a distinct geographical location (Newcastle) and each patient (PN), their isolated P. aeruginosa if present (BN), and serum (SN) were assigned the same number. Serum samples from eight patients with cystic fibrosis (SCF) and Pseudomonas colonization were from Birmingham. Colonization was defined by positive P. aeruginosa culture from sputum on at least two separate occasions.

Analysis and Manipulation of Serum.

Serum bactericidal assays were performed in triplicate using a modification of the method described MacLennan et al. (2010). In brief, bacteria were grown overnight in 5 ml of LB at 37° C. and resuspended in PBS to a final concentration of 107 CFU/ml; 10 μl was then mixed with 90 μl of undiluted human serum at 37° C. with shaking (180 rpm), and viable counts were determined. Serum mixing experiments were performed by first mixing the serum with either PBS, concentrated antibodies, other sera, unfiltered sol phase of sputum or sterile sol phase of sputum at the ratios described in text in a final volume of 90 μl before addition of bacteria. Killing was confirmed as caused by the activity of complement by 56° C. heat inactivating the serum as a control. Killing of Pseudomonas by washed peripheral blood cells was performed as previously described (Gondwe et al., 2010). In brief, bacteria were grown and resuspended in PBS as above before 10 μl was added to 90 μl of 1/10 dilution of sera (or PBS) for 20-min opsonization. At this point 10 μl this suspension was added to 90 μl of blood cells washed twice in RPMI. Samples were incubated on a rocker plate at 20 rpm at 37° C. and numbers of viable Pseudomonas were determined after 45, 90, and 180 min by serial dilution on Luria Bertani agar.

Complement deposition and antibody binding were quantified essentially as previously described (MacLennan et al., 2010). In brief, 5 μl Pseudomonas at an OD600=0.6 was mixed with 45 μl 10% serum (antibody determination) or undiluted serum (complement deposition) for 1 h at room temperature. After 3 washes with PBS a final incubation with FITC-conjugated anti-human immunoglobulin (Total IgG, IgG1, IgG2, IgG3, IgG4, IgA, IgM; Sigma-Aldrich) and anti-C1, C3, and C5b-9 (Dako). The C5b-9 antibody recognizes a neo-epitope on the MAC that only forms when the MAC assembles. After this final incubation, the cells were washed as before and analysed on a FACSAria II (BD). Total IgG subtype concentrations in sol phase sputum and serum samples were determined using the Human IgG Subclass Single Dilution Bindarid kit (Binding Site).

Fixation and preparation of Pseudomonas and sputum for cell imaging was performed as described previously (Leyton et al., 2011). In brief, poly L-lysine-coated coverslips loaded with fixed cells or a sputum streak were washed three times with PBS, and nonspecific binding sites were blocked for 1 h in PBS containing 1% BSA (Europa Bioproducts). Coverslips were incubated with 1:500 diluted serum or sol-phase sputum for 1 h, washed three times with PBS, and incubated for an additional 1 h with FITC-conjugated anti-human immunoglobulin (total IgG, IgG1, IgG2, IgG3, IgG4, IgA, IgM; Sigma-Aldrich). The coverslips were then washed three times with PBS, mounted onto glass slides, and visualized using either phase contrast or fluorescence using Leica DMRE fluorescence microscope (100× objective)-DC200 digital camera system.

Serum was fractionated with ultrafiltration columns (Vivascience) with 300, 100, and 30-kD size exclusion filters. In brief, 1 ml of serum was passed first through the 300-kD column as per manufacturer's instructions. Both the flow-through fraction and the retained fraction were diluted to a final concentration of 1 ml with PBS. The 1 ml flow-through fraction was then passed through the 100-kD column in the same way before the final passage through the 30-kD column. All four fractions (>300, 300-100, 100-30, and <30 kD) were brought to 1 ml final volume with PBS.

Antibodies were removed from serum using Protein A-Sepharose 4B, Protein G-Sepharose (GE Healthcare) or anti-human IgG2 monoclonal HP6200-Sepharose according to the manufacturer's instructions. All fractions retained were buffer exchanged into PBS to the desired volume before use in assays. Anti-LPS antibodies were removed from serum in the following manner. First, the LPS fraction was purified and quantified from the Pseudomonas using the method described below. The LPS preparation was diluted to 1 mg/ml and 1 ml mixed in microcentrifuge tube with 1 ml polymyxin-B agarose (Sigma-Aldrich) overnight at 4° C. The polymyxin B agarose has a binding capacity of 500 μg/ml so should be saturated with Pseudomonas LPS. The resin mix was then loaded onto the column and washed with 10 ml of 0.1 M ammonium bicarbonate buffer (pH 8.0). The serum was then passed over the column and washed with an additional 10 ml of buffer. Finally, bound antibody was eluted with a pH gradient of citric acid before buffer exchange into PBS.

P. aeruginosa biofilm formation was grown as described previously (Wells et al., 2008). In brief, 150 μl low-density P. aeruginosa culture was incubated in a 96-well plate overnight at 37° C. shaking. Nonadherent culture was then removed and replaced with 150 μl of serum or LB and incubated at 37° C. for 2 h. Supernatant was then removed and the biofilm stained with crystal violet. Biofilm intensity was measured at 595 nm. Analysis of bacterial fractions. Bacterial cell fractions were isolated and analyzed as previously described (Browning et al., 2003; Parham et al., 2004). In brief, outer membrane proteins were isolated by first separating the cell envelopes from the cytoplasm, after French pressure lysis of bacterial cells, by centrifugation (48,000 g for 60 min at 4° C.). The envelopes were retained and were resuspended in 3 ml of buffer (2% [vol/vol] Triton X-100, 10 mM Tris-HCl, pH 7.5) and incubated at 25° C. for 15 min to solubilize inner membrane components. Triton X-100-extracted envelopes were harvested by centrifugation at 48,000 g for 60 min at 4° C. and washed four times in 30 ml of 10 mM Tris-HCl, pH 7.5. Insoluble fractions were resuspended in 1 ml 10 mM Tris-HCl pH 7.5 and stored at −20° C.

LPS was isolated as previously described (Browning et al., 2003). In brief, Pseudomonas was grown overnight at 37° C. The equivalent of 1 ml of OD600=1 culture was spun and the pellet resuspended in 100 μl of lysing buffer (1 M Tris, pH 6.8, 2% SDS, and 4% 2-mercaptoethanol). The suspension was then boiled for 10 min, spun down, and supernatant was moved to a fresh Eppendorf. 5 μl of 5 mg/ml Proteinase K was added to each sample before incubation at 60° C. for 1 h. Finally, the LPS preparation was heated at 98° C. for 10 min and stored at ⊐20° C. LPS isolations were quantified by running the sample on an SDS-PAGE gel and comparing to five standards (10, 5, 1, 0.5, and 0.1 mg/ml) of commercially available Pseudomonas aeruginosa serotype 10 LPS (Sigma-Aldrich).

Bacterial cell fractions were visualized using SilverQuest kit (Invitrogen) or Western blotting (Raghunathan et al., 2011) using patient serum (1:200) and secondary antibody (1:5,000 alkaline phosphatase conjugated anti-human IgG, IgM or IgA; Sigma-Aldrich) before detection with nitro-blue tetrazolium and 5-bromo-4-chloro-31-indolyphosphate as the substrate.

Purification of LPS and/or O-Antigen

1 litre of Pseudomonas aeruginosa was grown overnight at 37° C. in LB

Take the equivalent of 1 litre of OD600: 2.5. (2.5/your OD600) and centrifuge this in order to obtain a bacterial pellet

### For Full LPS

Wash pellet twice in 20 mls PBS (pH=7.2) (0.15 M) containing 0.15 mM CaCl2 and 0.5 mM MgCl₂. Pellets were then resuspended in 20 ml PBS and sonicated for 10 min on ice.

Centrifuge the lysed culture for 5 minutes 3000 rcf and harvest supernatant.

### For Only O-Antigen

Resuspend pellet in 20 mls PBS+2% acetic acid

Boil this for 3 hrs, followed by centrifugation for 20 mins at 8500 rcf and harvest supernatant.

### Continue Below

100 ug/ml proteinase K added to 20 ml supernatant (FULL LPS or O-antigen).

Incubated this at 65° C. for one hour.

Add 40 ug/ml RNase and 20 ug/ml DNase in the presence of 1 μL/mL 20% MgSO₄ and 4 μL/mL chloroform and incubation was continued at 37° C. for 2 hrs.

An equal volume of hot (65-70° C.) 90% phenol (20 mls) was added to the mixtures followed by vigorous shaking at 65-70° C. for 15 min.

Cool the extracts on ice and centrifuge the falcons 8500×g for 15 min.

Supernatants (water phase) were transferred to 50 mL falcon centrifuge tubes.

20 ml of water added to phenol filled falcons again and remixed-respun-retake supernatant.

Resulting aqueous solution contains LPS or O-antigen.

For O-antigen, use tangential flow filtration (30 kDA filter) to condense and purify O-antigen in final volume 20 mls water.

Statistical Methods.

All experiments were performed at least three times unless otherwise stated. Correlation was determined using Spearman's rank and Pearson product-moment correlation coefficients. Statistical significance between patient groups was determined by Student's t test. Error bars represent ±1 standard error.

### Results

Impaired serum killing in bronchiectasis patients Historical data associated impaired serum-killing of P. aeruginosa with poor outcome in a patient with bronchiectasis (Waisbren and Brown, 1966). To explore if this is an isolated event or a more general phenomenon, we examined the serum sensitivity of P. aeruginosa isolates taken from 11 different patients with bronchiectasis and chronic Pseudomonas infection. Serum was collected from each patient and 20 healthy individuals. Each patient (P) and their isolated bacterium (B) and serum (S) were assigned the same number; patient P1, with serum S1, is colonized by P. aeruginosa B1. We found that eight patients had serum (S4-11) that could kill their cognate colonizing strain (B4-11), but three patients had serum (S1-3) that failed to kill their infecting strains (B1-3; FIG. 1 A). The bactericidal activity of the eight sera (S4-11) was inactivated by heat treatment, implying that serum killing was caused by the action of complement (unpublished data). The strains from patients with impaired bacterial killing were not innately resistant to killing, as sera from 20 healthy human controls (HCS) and sera from patients with bronchiectasis but without P. aeruginosa colonization (SN18-30) killed these three strains within 45 min (FIG. 1 B). Similar results were found for B2 and B3 (unpublished data). Next, we tested each patient's serum against all 11 of the P. aeruginosa isolates. We found that S1-3 could not kill B1-3 but could kill the P. aeruginosa strains from the other 8 patients (FIG. 1 C). In contrast, S4-11 could kill B1-3 (FIG. 1 D). This suggests the factors mediating resistance to serum killing are common to B1-3 and S1-3 but absent from the other strains and sera. We extended this analysis by testing sera (SN1-17) isolated from patients with bronchiectasis from a geographically distinct cohort who were colonized with P. aeruginosa. Three sera (SN1-3) failed to kill strain B1 but could kill strain B4, whereas the remainder (SN4-17) could affect serum-mediated killing of both B1 and B4 (FIGS. 1, E and F). A similar phenomenon was observed for a small sample of patients with cystic fibrosis (Table 1). Thus, ˜20% of the patients with bronchiectasis and P. aeruginosa infection had impaired serum killing of their strains, and the factor involved appeared to be specific both to the patient sera and the infecting P. aeruginosa strain.

### Impaired Serum Contains a Blocking Factor

We next explored whether the impaired serum killing results from an inhibitory factor present within the serum or from the lack of a serum component required for bactericidal activity. Specific anti-P. aeruginosa IgG, IgA, and IgM were present in the sera with impaired capacity to kill, at levels comparable to or greater than those in HCS that killed all the bacterial isolates (FIG. 2 A). Furthermore, IgG and complement components C1q, C3, and the C5b-9 membrane attack complex (MAC) were deposited on all strains (FIGS. 2, A and B). Antibody binding and complement deposition were confirmed by immunofluorescence microscopy (unpublished data). Thus, the impaired serum killing is not due to a lack of complement or antibody binding.

To determine if the lack of bacterial killing was due to a blocking factor in the serum, we mixed serum with impaired killing with HCS. Addition of HCS to S1-3 (50:50) did not restore serum killing, whereas HCS similarly diluted with PBS readily killed P. aeruginosa (FIG. 2 C). These data suggest that impaired serum-killing by S1-3 is caused by the presence of a factor inhibiting serum-mediated killing. In fact, complete killing by S1-3 was only restored when HCS represented 94, 70, and 80%, respectively, of the mixed sera (FIG. 2 D), indicating the patients serum had a potent capacity to inhibit killing.

### IgG Blocks the Ability for Serum to Kill Specific Pseudomonas Strains

We established that the impaired serum killing of patients' cognate Pseudomonas strains is due to a blocking factor in their serum. To identify the inhibitor, S1 was fractionated, based on molecular weight, and fractions were added to HCS. Inhibition was observed when the 100-300-kD fraction was added to HCS (FIG. 3 A). As this fraction contains IgG antibody, we investigated whether depleting antibody could restore bactericidal activity. Antibody was depleted by passing S1 over either a Protein A or a Protein G column, reducing total IgG titers ˜100-fold (FIG. 3 B). We found that the inhibitory serum S1, when depleted of antibody using either a Protein A or G column, could kill B1 within 180 min. Moreover, when mixed with HCS (50:50), B1 was rapidly killed indicating that the antibody-depleted serum no longer had the capacity to inhibit the bactericidal activity of HCS (FIG. 3 C). Importantly, antibodies eluted from the Protein A and G columns, in a volume equal to that of S1 originally applied to the column, inhibited the bactericidal activity of HCS, even when added at low concentrations (4-6%; FIG. 3 D). Similar observations were made for S2 and S3 (unpublished data). Due to the different antibody binding affinities of Protein A and G—protein G does not bind IgA or IgM), these findings suggest IgG is the blocking factor present in S1-3.

### IgG2 is the Inhibitory Factor in Impaired Serum

All of the initial cohort of 11 patients had normal proportions of the four IgG subclasses overall (Table 1); however, to determine if a specific IgG isotype could be responsible for the impaired killing of bacteria by serum seen in 3 patients, the titer of each IgG sub-class specific for P. aeruginosa was determined. Anti-P. aeruginosa IgG1 titers were not statistically different between impaired and normal killing sera groups (FIG. 4 A), and neither were levels of IgG3 and IgG4 (not depicted). In contrast, S1-3 had significantly (P<0.001) increased titers of anti-P. aeruginosa IgG2 compared with sera displaying normal bactericidal activity (FIG. 4 A). To test if IgG2 is the inhibitory factor, we purified IgG2 from S1 by passing the serum over an affinity column coated with a monoclonal antibody against human IgG2 (Jefferis et al., 1992). The IgG2-depleted flowthrough lost its inhibitory capacity. In contradistinction, IgG2 eluted from the column, in the same volume of serum loaded on the column, blocked the serum bactericidal activity of HCS (FIG. 4 B).

TABLE 1
Total antibody titers of brochiectasis patients

| Patient | IgG | IgA | IgM |
|---|---|---|---|
| P1 | 10.73 | 2.2 | 1.07 |
| P2 | 12.66 | 2.17 | 0.82 |
| P3 | 10.69 | 4.49 | 1.05 |
| P4 | 11.81 | 4. [symbol] | 1.29 |
| P5 | 11.18 | 1.51 | 4.19 |
| P6 | 11.91 | 3.4 | 1.64 |
| P7 | 16.64 | 10.37 [symbol] | 0.73 |
| P8 | 16. [symbol] | 3 | 3.08 |
| P9 | 14.5 | 3.59 | 1.1 [symbol] |
| P10 | 13.8 [symbol] | 2.78 | 0.99 |
| P11 | 12.5 | 1.87 | 0.77 |
| Normal range | 6.0-16.00 | 0.8-4.0 | 0.50-2.00 |

*No [symbol] determined by [symbol] [symbol]
indicates data missing or illegible when filed

### LPS is the Target of the Inhibitory IgG2

To determine if the inhibitory IgG2 antibody targeted a specific bacterial factor, we performed Western immunoblotting of outer membrane protein and polysaccharide fractions with patient serum and anti-human IgG. S1 contained antibodies that recognized proteins from all strains (FIG. 5 A). In contrast, the serum only recognized the O-antigen side chains of LPS from B1-3 and did not recognize O-antigen of strains from patients without impaired serum killing (FIG. 5 B). Similar results were obtained for S2 and S3, whereas HCS had no detectable anti-O-antigen antibody to B1-3 (FIG. 5 C). SDS-PAGE and silver staining of LPS fractions revealed that the strains B1, B2, and B3 produced significant amounts of long-chain O-antigen but the other strains did not (FIG. 5 D). By binding the LPS isolated from B1 to an ELISA plate we determined that the patients with impaired serum-mediated bacterial killing had high levels of anti-LPS IgG by ELISA (FIG. 5 E). To test if the level of anti-LPS antibody is responsible for this effect, rather than simply the presence of anti-LPS antibodies per se, we purified anti-LPS antibodies from S4, a serum that has normal bactericidal activity. The eluted antibodies, concentrated 10-fold on the column (FIG. 5 F), inhibited the bactericidal activity of HCS in a dose-dependent manner (FIG. 5 G), indicating the titer of anti-LPS antibody in serum is critical for inhibition.

### Antibodies Against O-Antigen, but not Lipid A or Core, Inhibit Serum Killing

The three strains that could not be killed by serum containing blocking IgG2 possessed high amounts of O-antigen. These observations suggest the long-chain O-antigen of LPS is the target of inhibitory antibody. To test this, LPS purified from B1 was immobilized on a polymyxin-B agarose column and S1 was passed through the column to remove antibody specific to the LPS (FIG. 6 A). The recovered flow-through fraction was now able to kill B1 and no longer inhibited the killing activity of HCS (FIG. 6 B). Conversely, anti-LPS antibody eluted from the column inhibited the bactericidal activity of HCS in a dose-dependent manner (FIGS. 6, A and C). Immunofluorescence microscopy revealed that the flow-through fraction lacked detectable anti-P. aeruginosa IgG2 (unpublished data). In contrast, anti-P. aeruginosa IgG1 remained detectable in the serum depleted of anti-LPS antibody (FIG. 6 A).

All P. aeruginosa strains contain lipid A and core oligosaccharide of LPS. Consequently, we depleted S1 of antibody to lipid A and the core elements by passing S1 over a polymyxin B column on which LPS isolated from B4, which lacks O-antigen, was immobilized (FIG. 6 A). The flow-through antibody had a 30-fold lower level of binding to lipid A and core oligosaccharide compared with both the native serum and the antibody eluted from the column (FIG. 6 A). However, the flow through from this column still recognized O-antigen-containing LPS purified from B1 at a level similar to native S1 (FIG. 6 A). This flow-through inhibited the killing of B1 by HCS, but the antibody recognizing lipid A and core oligosaccharide eluted from the column did not (FIG. 6 D).

### The Role of Inhibitory Antibodies in the Lung

The role of serum-mediated killing in controlling bacterial growth during lung infection is not widely recognized. However, previously high levels of antibody were shown to be present in the lungs of patients suffering from bronchiectasis (Hill et al., 1998). We hypothesized that patients with impaired immunity and P. aeruginosa colonization would have high titers of IgG2 present in the lung. To confirm this, the sol phase of sputum from P2 (impaired killing) and P4 (normal killing) was harvested and the levels of IgG1 and IgG2 were measured. P2 sol phase sputum had S12 and 220 mg/liter IgG1 and IgG2, respectively, whereas P4 sol phase contained 315 mg/liter IgG1 and 158 mg/liter IgG2.

Having demonstrated the presence of antibody in the lung, we next sought to determine whether this antibody played a role in protecting bacteria from serum-mediated killing in vivo. To establish this, we first investigated whether P. aeruginosa was opsonized by antibody in vivo. Immunofluorescence microscopy of sputum smears revealed bacteria present within the sputum were labeled with anti-human IgG2-FITC (FIG. 7 A). Additional investigations revealed cultured bacteria could also be labeled with IgG2 when opsonized with 1:200 sol phase sputum (FIG. 7 A). To confirm that this opsonization protects bacteria from complement-dependent killing, we explored whether HCS could kill bacteria from sputum. The unfiltered sol phase of sputum from P2, containing opsonized bacteria, was mixed 50:50 with HCS; however, there was no reduction in bacterial numbers over 180 min of incubation (FIG. 7 B). To confirm this phenomenon, B1 was incubated with a mixture of HCS and sterile sol-phase from P2. No complement-dependent killing was observed over 180 min. In contrast, B1 incubated with a mixture of HCS and sterile sol-phase from P4 was rapidly killed within 45 min (FIG. 7 B).

Opsonization is important for cell-mediated killing, which is known to play a vital protective role within the lung (Whitters and Stockley, 2012). We hypothesized that inhibitory antibodies may also play a role in cell-mediated killing. Thus, we investigated killing of B1 and B4 by washed peripheral blood cells. B1 opsonized with HCS was rapidly killed on incubation with peripheral blood cells. Similarly, opsonization of B4 with either HCS or S1 led to rapid killing of the bacteria. In contrast, B1 opsonized with S1 was not killed (FIG. 7 C).

These data suggest an important role for inhibitory antibody in protecting bacteria within the lung from immunemediated clearance. However, it is accepted that P. aeruginosa resides in a biofilm within the lung. Therefore, we investigated the effect of serum on an established biofilm. B1 forms a thick biofilm in a 96-well plate over 24 h. Incubation of the B1 biofilm with HCS and S4 sera for 2 h drastically reduced the amount of biofilm. In contrast, S1 had no effect on the amount of biofilm over a similar period (FIG. 7 D).

Patients with Inhibitory Antibodies have Worse Lung Function

The results of the aforementioned in vivo and in vitro studies suggest that the presence of inhibitory antibody may have clinical relevance. Thus, we sought to determine whether patients with bronchiectasis and inhibitory levels of anti-LPS IgG2 antibody had more marked disease severity than those patients whose serum could mediate killing. We used forced expiratory volume in 1 s (FEV1) as a measure of lung function. Individuals colonized with P. aeruginosa who also possessed inhibitory antibody had poorer lung function when compared with individuals colonized with P. aeruginosa whose serum displayed normal killing (P<0.002) and patients with bronchiectasis who were not colonized with P. aeruginosa (P<0.05; FIG. 8 A. This indicates the impaired capacity to kill bacteria has clinical consequences. Interestingly, a similar proportion of patients from two different cohorts displayed IgG2-mediated inhibition of serum killing, suggesting there may be an underlying genetic, rather than acquired, basis for an elevated response.

### IgA Directed Towards O-Antigen is Also Elevated in Some Patients

We next sought to determine if IgA can target O-antigen. We observed IgA binding in 2 out of the three patients tested (FIG. 9 A). Western blot analysis (FIG. 9 B) demonstrated that the IgA is specific for the same O-antigen as the IgG2 (see S2, which has high LPS-specific IgG2 and IgA response). In contrast, no anti-LPS IgA was detected in HCS.

It is possible that IgA may also be involved in inhibition due to the data showing raised levels of IgA to the same epitope as the inhibitory IgG2.

### DISCUSSION

Antibody is usually associated with protection against infectious disease. In contrast, antibody-dependent enhancement of infection is seen for some microbial organisms, most notably viruses such as dengue fever (Halstead and O'Rourke, 1977), but to a lesser extent parasitic organisms such as leishmaniasi (Halstead et al., 2010). In the case of dengue fever, circulating antibodies bind to the newly infecting virus but do not neutralize infection. Instead, these antibodies enhance viral entry via efficient interaction of the virus-antibody complex with Fc receptors (Halstead et al., 2010; Flipse et al., 2013). However, the action of antibody in exacerbating bacterial infectious disease is less well understood. Our results indicate that in patients with bronchiectasis, who are chronically colonized with P. aeruginosa, the presence of high titers of IgG2 antibodies specific for the O-antigen of LPS impairs serum-mediated killing of the infecting strain and is associated with a poorer lung function. Here, we describe antibody-dependent enhancement of bacterial infection and demonstrate the mechanism is different to that for dengue.

Lack of serum bactericidal activity against P. aeruginosa has previously been noted for patients with CF (Waisbren and Brown, 1966; Guttman and Waisbren, 1975). Moreover, increased anti-LPS antibody titers have been noted in CF patients chronically infected with P. aeruginosa (Fick et al., 1986). Separately, high levels of IgG3 and IgG2 specific for lipid A and O-antigen were shown to correlate with deteriorating pulmonary function (Kronborg et al., 1993). In contrast, our data demonstrate that in bronchiectasis patients, high titers of IgG2 specific for the O-antigen of LPS are sufficient to impair serum-mediated killing of P. aeruginosa. Importantly, high titers of IgG2 in the sputum are associated with phenotypes within the lung, including opsonization of infecting bacteria, inhibition of cell mediated killing, and lack of biofilm clearance.

The biological properties of IgG2 may be a factor in its role as an inhibitor of serum- and/or cell-mediated killing. Switching to IgG2 is particularly associated with responses to bacterial polysaccharides (Siber et al., 1980) but, in contrast to IgG1 and IgG3, the C1q-binding sites on IgG2 are frequently not exposed on antigen binding (Brüggemann et al., 1987; Schroeder and Cavacini, 2010). IgG2 also binds to only one class of Fc⊐R (FcγRII), whereas other IgG classes bind multiple classes (Normansell, 1987; Schroeder and Cavacini, 2010). Indeed, IgG2 antibodies have been seen to exert antiphagocytic effects on P. aeruginosa (Hornick and Fick, 1990). However, we hypothesize that anti-O-antigen IgG2 inhibits killing of the P. aeruginosa strains by a mechanism similar to that recently described for nontyphoidal Salmonella enterica infection in some HIV-infected Malawian adults (MacLennan et al., 2010).

Thus, inhibitory IgG2 antibodies bind O-antigen, a target distal on the LPS molecule, and exert their inhibitory effect either by activating and depositing complement away from the bacterial membrane and preventing MAC insertion or by blocking access of protective antibody (Brown et al., 1983; Moffitt and Frank, 1994; MacLennan et al., 2010). However, we have yet to establish whether low titers of anti-O-antigen IgG2 can promote bacterial killing without the addition of other protective antibodies (Taborda et al., 2003). Notably, in the Salmonella study, although IgG was found to be inhibitory in the serum, the specific isotype conferring inhibition was not identified. Furthermore, in the current study, the impaired serum killing is not associated with HIV infection or an immunocompromised state.

Our findings have significant implications for vaccine design. Currently, LPS is thought to be an optimal target for protective antibodies. Three O-antigen-based vaccines against P. aeruginosa, Pseudogen, PEV-01, and Aerugen, have reached phase II or III trials (Pennington et al., 1975; Langford and Hiller, 1984; Cryz et al., 1997). However, two vaccines resulted in worse clinical status in the vaccinated group and the third trial was suspended (Cryz et al., 1989; Döring and Pier, 2008).

These studies have not detailed the IgG subclasses induced in response to the vaccine. The current study provides a potential mechanistic basis for the failure of these vaccines strategies. It indicates that candidate O-antigen polysaccharide-based vaccines may elicit imbalanced anti-O-antigen (IgG2 dominant) antibody induction, rendering the vaccine ineffective while increasing the susceptibility to life-threatening P. aeruginosa infections. Furthermore, historical reports of the association of impaired serum killing with other bacterial infections suggest this mechanism may be common for a wide variety of Gram-negative bacterial infections (Waisbren and Brown, 1966). Importantly, understanding the impact elevated levels of IgG2 have on infections could provide opportunities to attenuate disease in several clinical settings.

Enzyme-Linked Immunosorbent Assay (ELISA)—Anti-O-Antigen from P. aeruginosa

The ELISA method adopted for this protocol allows the measurement of natural levels of IgA, IgM and IgG antibodies against P. aeruginosa LPS present within serum samples. By performing a serial dilution of each serum sample abolishes the need for a standard reference control to compare samples.

### Method:

### Day 1

Coating of ELISA Plates with O-Antigen
1. Prepare P. aeruginosa O-antigen (prepared as described above) mix in coating buffer (Na₂CO₃ (Sodium Carbonate)—1.95 g (0.015M; NaHCO₃ (Sodium Bicarbonate)—2.93 g (0.035M) dissolve together in 1 litre distilled water and pH to 9.6) to a final concentration of 1 μg/ml sufficient for 100 μl/well (10 ml/plate)
2 Add 100 μl/well, cover with parafilm and the lid, place plates in a humid chamber and incubate overnight at 4° C.
   NOTE: This overnight step can be eliminated and replaced with ˜1 hour at 37° C. in humid chamber if short on time

### Day 2

Blocking of ELISA Plates with Bovine Serum Antigen

Begin by preparing the wash buffer (0.1M PBS, pH 6.8, 0.05% Tween 20); blocking buffer (0.1M PBS, pH 6.8, 1% Bovine serum albumin); and dilution buffer (0.1M PBS, pH 6.8, 0.05% Tween 20, 1% Bovine serum albumin)
1. Pour/shake off overnight coat and wash plates with wash buffer (3×) by immersing plates completely in the buffer and dry by knocking on the bench onto a paper towel
2. Add 200 μl/well blocking buffer, place plates in a humid chamber and incubate 1-1½ hours at 37° C.
   NOTE: Plates can also be frozen at −20° C. in blocking buffer (immediately following addition of blocking buffer) for long-term storage.
   NOTE: Other alternatives include 1. coat the plates for ˜1 hour, wash 3×, then add blocking buffer and freeze immediately, or 2. coat the plates for ˜1 hour, wash 3×, then add blocking buffer and incubate in humid chamber overnight at 4° C.

### Binding of Test Serum Antibodies to LPS

1. Thaw test serum and keep on ice until required
2. Wash plates with wash buffer (3×)
3. Add the required volume of dilution buffer to the plates (see FIG. 1), then add the required volume of test serum to rows 1 and 7 of the 96-well ELISA plates (for an example using a starting dilution of 1:20 and a 3-fold dilution series)
4. Mix by pipetting up and down then perform a dilution series down 6 rows of the 96-well ELISA plates, ensuring the volume transferred down the plate is discarded from the final wells of the dilution series, leaving a total volume of 100 μl/well—this is important as the ELISA works on the principle of measuring the absolute amount of antibody available to bind, not on the overall concentration
5. Place plates in a humid chamber and incubate 1 hour at 37° C.

### Secondary Antibody Binding to Test Serum Antibodies

1. Prepare secondary antibodies in dilution buffer (10 ml/plate):

| | |
|---|---|
| Anti-human IgG (1,2,3,4)-AP (Cat#2040-04) - | 1:2000 |
| Anti-human IgM-AP (Cat#2020-04) - | 1:2000 |
| Anti-human IgA-AP (Cat#2050-04) - | 1:2000 |

Wash Plates with Wash Buffer (0.1M PBS, pH 6.8 0.05% Tween 20) (3×)
1. Add 100 μl 1:2000 secondary antibody/well to the appropriate plate, place in a humid chamber and incubate 1 hour at 37° C.

### Determination of Test Serum Antibody Concentrations Through Measurement of Signal

1. Prepare SIGMAFAST™ p-Nitrophenyl phosphate substrate in distilled water—1×Tris buffer tablet and 1×PNPP tablet/20 ml d.H₂O
   NOTE: These tablets take a long time to dissolve—add the Tris tablet after the addition of secondary antibody, then the PNPP approximately 10 minutes before the end of the secondary antibody incubation
2. Add 100 μl substrate/well and incubate plates at room temperature (or at 37° C. if reaction is slow), measuring the OD at 405 nm at regular intervals

### Multiplexed Assay

Coating with Multiple O-Antigen Serotypes
1. Prepare multiple P. aeruginosa O-antigen serotypes (prepared as described above) and mix 2 or more of these together appropriate buffer in equal ratios to a final total concentration of 5 μg/ml
   a. If using an ELISA plate—add 100 μl/well, cover with parafilm and the lid, place plates in a humid chamber and incubate overnight at 4° C.
   b. Other methods and products can be used to immobilise the O-antigen. The rest of the protocol is designed for use in 96 well plates—however this protocol can be adjusted for any other detection methods using patients sera as primary antibody and an appropriate IgG2-specific secondary antibody for detection.

### Blocking

Begin by preparing the wash buffer (0.1M PBS, pH 6.8, 0.05% Tween 20); blocking buffer (0.1M PBS, pH 6.8, 1% Bovine serum albumin); and dilution buffer (0.1M PBS, pH 6.8, 0.05% Tween 20, 1% Bovine serum albumin)
1. Remove overnight coat and wash plates with wash buffer (3×)
2. Add 200 μl/well blocking buffer, place plates in a humid chamber and incubate 1-1½ hours at 37° C.

### Binding of Test Serum Antibodies to LPS

1. Thaw test serum and keep on ice until required. You can also use positive and negative control sera as well as pre-defined diluted controls for a standard curve.
2. Wash plates with wash buffer (3×)
3. Dilute patient sera the appropriate amount in dilution buffer to a final volume of 100 ul (if doing one sample—but duplicates/triplicates could be used). Currently a dilution of 1:100 is used however lower/higher dilutions could be used if required.
4. Add 100 ul of the diluted patient sera to a single well in the plate. You can at this point add more to other wells for replicates
5. Place plates in a humid chamber and incubate 1 hour at 37° C.

### Secondary Antibody Binding to Test Serum Antibodies

2. Prepare secondary antibodies in dilution buffer (10 ml/plate):

| | |
|---|---|
| Anti-human IgG2-AP (or other suitable conjugate) - | 1:2000 |

Wash Plates with Wash Buffer (3×)
2. Add 100 μl 1:2000 secondary antibody/well to the appropriate plate, place in a humid chamber and incubate 1 hour at 37° C.

### Determination of Test Serum Antibody Concentrations Through Measurement of Signal

3. Prepare SIGMAFAST™ p-Nitrophenyl phosphate substrate in distilled water—1×Tris buffer tablet and 1×PNPP tablet/20 ml d.H₂O (or other suitable substrate)
4. Add 100 μl substrate/well and incubate plates at room temperature, measuring the OD at 405 nm after 1 hour. (or measure depending on the conjugate/substrate choice at appropriate time)
5. The response in each individual well reflects the amount of anti-O-antigen IgG2. A cutoff will be determined where in samples with a response above the cutoff have inhibitory IgG2 concentrations and samples below that cutoff will not have sufficient antibody to cause inhibition. This cutoff needs to be determined using the standard curve of controls and will change depending on how many O-antigens are used in the multiplexed assay.

1. A method for detecting the presence or elevated level of IgG2 specific for O-antigen from Gram-negative bacteria, in a sample from a subject, the method comprising providing a binding agent specific for said IgG2, contacting the binding agent with the sample, allowing the binding agent and IgG2 to form a complex and thereafter directly or indirectly detecting the complex.

2. The method according to claim 1 wherein the agent specific for said IgG2 is O-antigen, or a IgG2 specific fragment thereof.

3. The method according to claim 1 for detecting the presence and/or initial colonization of Gram-negative bacteria in a patient.

4. The method according to claim 1 for detecting severe or worsening disease.

5. The method according to claim 4 for detecting a worsening airway, lung and/or bronchiolar tree condition.

6. The method according to claim 5 wherein the disease is bronchiectasis, such as non-cystic fibrosis bronchiectasis.

7. The method according to claim 5 wherein the disease is cystic fibrosis.

8. The method according to claim 1 wherein the level of O-antigen specific IgG2 is capable of inhibiting immune-killing of O-antigen containing bacteria.

9. The method according to claim 1 wherein the O-antigen is from any Gram negative bacterial species which are typically associated with infection in humans or animals, such as Escherichia coli (E. coli), Salmonella, Shigella, Enterobacteriaceae, Pseudomonas (especially Pseudomonas aeruginosa), Moraxella, Helicobacter, Stenotrophomonas, Bdellovibrio, Legionella, Neisseria, Ralstonia, Klebsiella, Acinetobacter, Proteus, and Serratia.

10-11. (canceled)

12. A method of determining the efficacy of treatment for a smooth Gram negative infection in an subject, comprising determining in samples from the subject, whether the levels of smooth Gram-negative bacteria which express O-antigen capable of binding lgG2 specific for said O-antigen has decreased after the treatment.

13-26. (canceled)

27. The method according to claim 1 wherein the level of O-antigen and/or IgG2 is detected by way of a radioimmunoassay (RIA), enzyme-linked immunosorbent assay (ELISA), Western blotting, flow cytometry, electrochemiluminescent assays, plasmon and surface enhanced resonance assay, a histological technique, or mass spectrometry technique.

28. The method according to claim 27 wherein the level of O-antigen and/or IgG2 is detected by an immunological method, such as a competitive or non-competitive immunoassay, preferably using a solid-phase antibody, an ELISA or ELISPOT assay.

29. An isolated O-antigen for use in a method according to claim 1.

30. A mixture comprising 2, 3, 4, 5, 6, 7, 8, 9 or more isolated separate O-antigens of different serotype of O-antigens according to claim 29.

31. The isolated O-antigen according to claim 29 bound to a suitable substrate such as sepharose, polylysine, polymyxin B, magnetic beads or plastics material.

32. A kit comprising the isolated O-antigen according to claim 29.

33. A method of obtaining an isolated O-antigen(s), the method comprising:
providing a bacterial strain or strains which express O-antigen capable of specifically binding an inhibitory immunoglobulin(s), growing the bacterial strain(s) and obtaining the isolated O-antigen.

34. The method according to claim 33 wherein the O-antigen may be purified from the bacterial strain(s) and may be free from cell wall components and/or LPS.

35. The method according to claim 34, wherein the purification includes the use of acetic acid.

36. The method according to claim 33 wherein at least 2, 3, 4, 5, 6, 7, 8 or 9 different serotyped O-antigens are obtained or purified.
